(19) United States
(12) Patent Application Publication
(10) Pub. No.: US 2006/0089322 A1
(43) Pub. Date: Apr. 27, 2006

(54) Antisense oligonucleotides for identifying drug targets and enhancing cancer therapies

(76) Inventors: Mark Vincent, London (CA); Donald James Koropatnick, London (CA); Randall Berg, London (CA)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 10/513,255

(22) PCT Filed: May 1, 2003
(86) PCT No.: PCT/CA03/00610

§ 371 Date: Sep. 12, 2005

(30) Foreign Application Priority Data
May 1, 2002 (CA) 2 384 447

(51) Int. Cl. A61K 48/00; A61K 31/7072; A61K 31/513; C12Q 1/68; C07H 21/04

(52) U.S. Cl. 514/44; 514/49; 514/269; 536/23.2; 435/6

(57) ABSTRACT

The present invention provides antisense oligonucleotides useful for identifying drug targets for cancer therapies and for enhancing current cancer therapies. The oligonucleotides of the invention are complementary to thymidylate synthase mRNA and affect expression of at least one other gene. For the enhancement of cancer therapies, such antisense oligonucleotides can be used in conjunction with standard chemotherapeutic agents in order to target thymidylate synthase, as well as other appropriate targets. The antisense oligonucleotides and the methods of the invention constitute improved antisense therapies with application to a variety of cancers.

### FIELD OF THE INVENTION

The present invention pertains to the field of antisense oligonucleotides in cancer therapies.

### BACKGROUND

The use of antisense oligodeoxynucleotides (ODNs) as therapeutic molecules is known. Several antisense ODNs targeting a variety of molecules have been shown to have antiproliferative effects against neoplastic cells in vitro and in vivo (Gewirtz, 2000, J. Clin. Oncol. 18:1809-1811), and several have demonstrated anti-tumour activity and limited toxicity in Phase I clinical trials (Smith and Wickstrom, 2000, Methods Enzymol. 314:537-580).

There are a number of proteins that have been implicated in cancer and, as a result, have been targeted by cancer therapies using standard chemotherapeutics. An example is thymidylate synthase (TS), which is an essential enzyme in de novo production of thymidylate (Carreras and Santi, 1995, Annu. Rev. Biochem. 64:721-762). Due to the crucial role of TS in DNA synthesis and cell proliferation, it has been an important target for cancer chemotherapy for many years (Danenberg, 1977, Biochim. Biophys. Acta 473:73-92; Danenberg et al., 1999, Semin. Oncol. 26:621-631).

Chemotherapeutics that inhibit TS, such as 5-fluorouracil (5-FU) and its variants, have become integral drugs in standard treatments for colorectal cancer (Papamichael, 1999, Oncologist. 4:478-487). Raltitrexed (Tomudex®) and pemetrexed (Alimta®) are other TS inhibiting chemotherapeutics with a potential role in a range of cancers including mesothelioma. Although reasonably successful in clinical use, these drugs suffer from problems of dose-limiting toxicity and outgrowth of resistant cells, motivating the continued search for alternative treatments, such as antisense ODNs that target and impact upon the expression of TS mRNA (U.S. Pat. No. 6,087,489; International Patent Applications WO 99/15648 and WO 98/49287). A specific antisense oligonucleotide targeting the 3′-untranslated region of TS mRNA has been shown to down-regulate the expression of TS, inhibit neoplastic cell proliferation (Berg et al., 2001, J. Pharmacol. Exp. Ther. 298:477-484) and enhance the cytotoxicity of certain TS-targeting drugs in HeLa cells (Ferguson, et al., 1999, Br. J. Pharmacol. 127:1777-1786). More recently, the use of this antisense oligonucleotide to increase the sensitivity of cells that over-express TS to 5-FUdR has been demonstrated (Ferguson, et al., 2001, Br. J. Pharmacol. 134:1437-1446).

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide antisense oligonucleotides for identifying drug targets and enhancing cancer therapies. In accordance with an aspect of the present invention, there is provided a composition comprising: an antisense oligonucleotide, or analogue thereof, comprising at least 7 consecutive nucleotides complementary to a thymidylate synthase mRNA; a fluoropyrimidine-based chemotherapeutic, and optionally a pharmaceutically acceptable carrier or diluent, wherein said antisense oligonucleotide or analogue thereof, inhibits expression of a thymidylate synthase gene and modulates the expression of at least one other gene.

In accordance with another aspect of the invention, there is provided a use of an antisense oligonucleotide, or analogue thereof, comprising at least 7 consecutive nucleotides complementary to a thymidylate synthase mRNA to sensitise neoplastic cells to a chemotherapeutic agent, wherein said antisense oligonucleotide, or analogue thereof, inhibits expression of a thymidylate synthase gene and modulates the expression of at least one other gene.

In accordance with another aspect of the invention, there is provided a use of an antisense oligonucleotide, or analogue thereof, comprising at least 7 consecutive nucleotides complementary to a thymidylate synthase mRNA in combination with one or more chemotherapeutic agent in the treatment of cancer in a mammal in need thereof, wherein said antisense oligonucleotide, or analogue thereof, inhibits expression of a thymidylate synthase gene and modulates the expression of at least one other gene, and wherein said one or more chemotherapeutic agent is used at less than standard dosage.

In accordance with another aspect of the invention, there is provided a use of an antisense oligonucleotide, or analogue thereof, comprising at least 7 consecutive nucleotides complementary to a thymidylate synthase mRNA to increase the bioavailability of a fluoropyrimidine-based chemotherapeutic in a mammal in need thereof, wherein said antisense oligonucleotide, or analogue thereof, inhibits expression of a thymidylate synthase gene and modulates the expression of at least one other gene.

In accordance with another aspect of the invention, there is provided a use of an antisense oligonucleotide, or analogue thereof, comprising at least 7 consecutive nucleotides complementary to a thymidylate synthase mRNA to decrease one or more dose-limiting toxicities of a fluoropyrimidine-based chemotherapeutic in a mammal in need thereof, wherein said antisense oligonucleotide, or analogue thereof, inhibits expression of a thymidylate synthase gene and modulates the expression of at least one other gene.

In accordance with another aspect of the invention, there is provided a use of an antisense oligonucleotide, or analogue thereof, comprising at least 7 consecutive nucleotides complementary to a thymidylate synthase mRNA to potentiate the effect of a dihydropyrimidine dehydrogenase inhibitor in a mammal in need thereof, wherein said antisense oligonucleotide, or analogue thereof, inhibits expression of a thymidylate synthase gene and the expression of a dihydropyrimidine dehydrogenase gene.

In accordance with another aspect of the invention, there is provided a use of an antisense oligonucleotide, or analogue thereof, comprising at least 7 consecutive nucleotides complementary to SEQ ID NO: 2 to inhibit the expression of a dihydropyrimidine dehydrogenase gene in a mammal in need thereof.

In accordance with another aspect of the invention, there is provided a method of screening for potential drug targets for cancer therapy comprising the steps of: contacting a first population of cancer cells with an antisense oligonucleotide, or analogue thereof, comprising at least 7 consecutive nucleotides complementary to a thymidylate synthase mRNA; isolating mRNA from said cancer cells to provide a treated mRNA sample; isolating mRNA from a second control population of cells to provide a control mRNA sample; conducting gene expression assays using said treated mRNA sample and said control mRNA sample to determine genes whose expression is modulated in the first population of cancer cells, thereby identifying

### BRIEF DESCRIPTION OF THE FIGURES

FIG. 1 depicts a schematic of thymidylate synthase (TS) as a target for chemotherapeutic intervention in cancer. Reductive methylation of dUMP, the reaction catalysed by thymidylate synthase (TS), is the only de novo source of dTMP necessary for DNA replication and repair. Cancer chemotherapy drugs such as 5-fluorouracil (5-FU) and raltitrexed (RTX) target the binding sites on TS for dUMP and folate, respectively. TS inhibition by these drugs leads to unbalanced nucleotide pools, impaired DNA synthesis cell cycle arrest in early S phase, and apoptosis.

FIG. 2 depicts a schematic of the mechanism by which TS upregulation contributes to anticancer drug resistance. TS protein binds to its mRNA, inhibiting translation. Substrate or inhibitor addition results in dissociation of the TS protein-mRNA complex, increased translation and TS protein levels, and reduced efficacy of anti-TS drug treatment. TS overexpression can mediate drug resistance, and high tumour TS levels is a prognostic for poor response to fluoropyrimidine and antifolate therapy.

FIG. 3 illustrates one mechanism by which TS antisense oligodeoxynucleotides target TS. In general, antisense drugs target specific nucleotide sequences in mRNA molecules. Hybridization can inhibit translation initiation or ribosomal processivity or, in the case of oligodeoxynucleotides (ODNs), initiate target mRNA degradation by recruitment of RNase H and subsequent exonuclease action (FIG. 3A). Fully phosphorothioated 20-mers, with 2′-methoxyethoxy modifications on each of the 6 terminal riboses (5′- and 3′-ends) were provided by Isis Pharmaceuticals. These modifications improve ODN delivery, stability and hybridization with target mRNA. The middle 8 nucleotides are unmodified, providing optimum RNAse H activity (FIG. 3B). FIG. 3C illustrates examples of the location and sequence of ODNs targeting TS.

FIG. 4 depicts a schematic of the downstream effects of TS-targeted ODNs.

FIG. 5 illustrates the effects of TS antisense ODN treatment on gene expression in HeLa cells using microarray analysis and RT-PCR. FIG. 5A shows the results on gene expression in HeLa cells exposed to TS antisense ODN treatment at 24 versus 48 hours. FIGS. 5B to E show that TS antisense ODN SEQ ID NO: 2 treatment repressed expression of at least 12 genes. FIG. 5F shows that at 8 and 16 hours following treatment with ODN SEQ ID NO: 2 many other genes besides the ones described above were either repressed or induced. A time course of the effects of ODN SEQ ID NO: 2 on HeLa cell TS mRNA levels is shown in FIG. 5G.

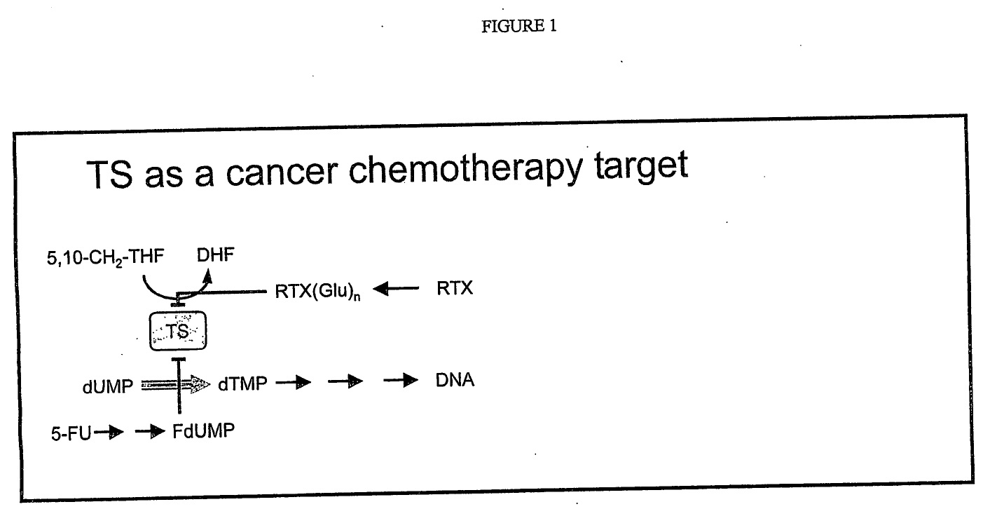

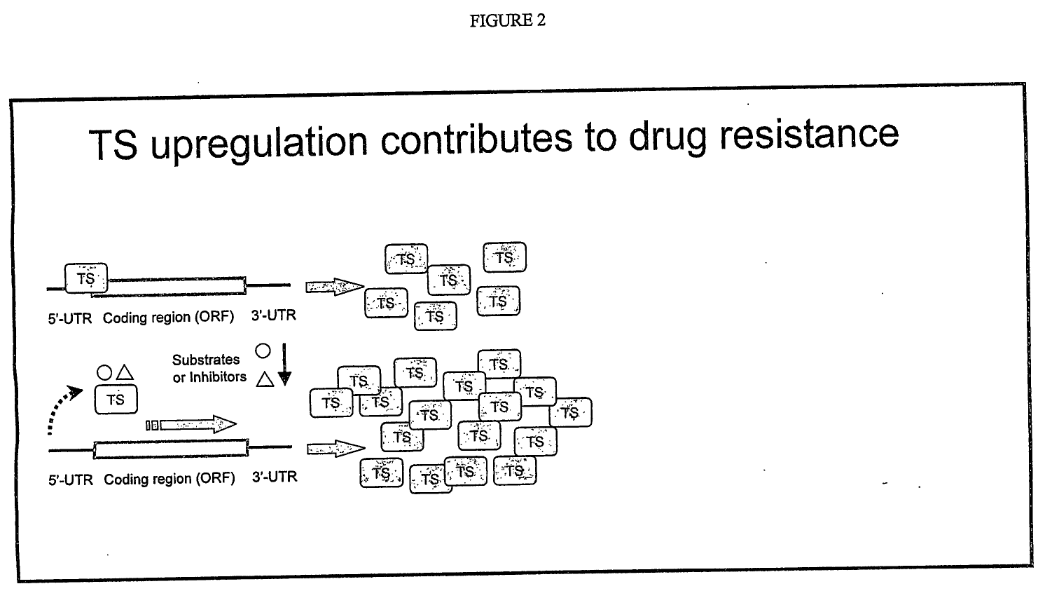

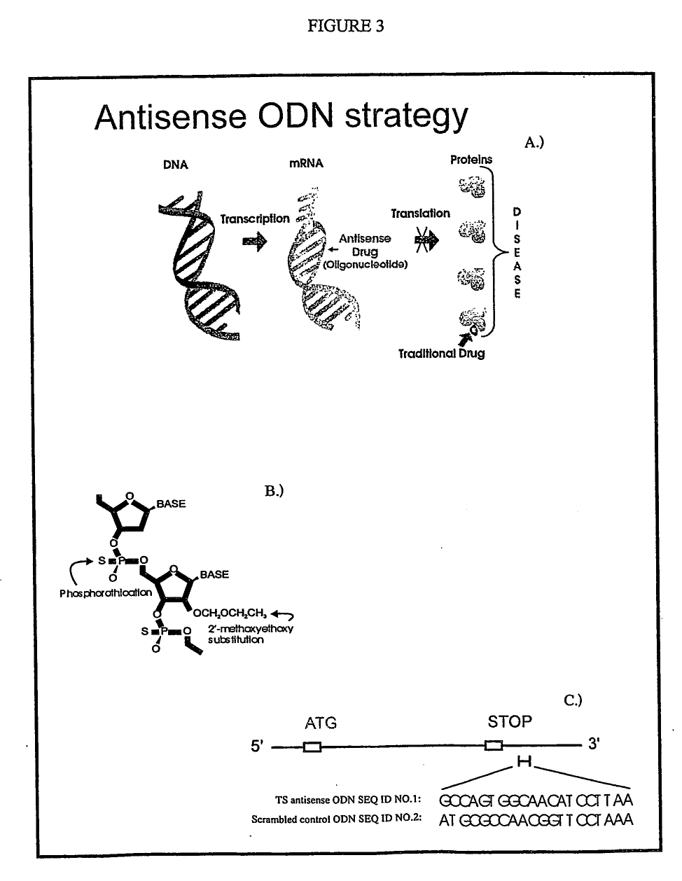

### DETAILED DESCRIPTION OF THE INVENTION

The present invention provides strategies for the development and design of efficacious antisense ODNs that can be used to modulate expression of a thymidylate synthase (TS) gene and affect expression of at least one other target gene. This represents a novel strategy to developing new cancer therapies or improving known cancer therapies with a wide range of applications.

The invention thus provides for a method of identifying genes that are, or whose gene products are, potential targets for cancer therapies using antisense oligonucleotides (ODNs) that are complementary to a portion of a TS gene.

The invention further provides for the use of antisense ODNs that are complementary to a portion of a TS gene and that modulate expression of TS and at least one other gene in combination with one or more chemotherapeutic agents in the treatment of cancer. In accordance with one embodiment of the present invention, the antisense ODNs enhance the effect of standard doses of the chemotherapeutic and/or minimise one or more side-effects associated with the use of the chemotherapeutic.

In accordance with another embodiment of the invention, the antisense ODN inhibits expression of TS and expression of dihydropyrimidine dehydrogenase (DPD). Such antisense ODNs can be used to enhance the effect of fluoropyrimidine-based drugs by minimising their catabolism in vivo.

In another embodiment of the present invention, the antisense ODN inhibits expression of TS and expression of protein tyrosine phospatase-1B.

Definitions

Unless defined otherwise, all technical and scientific terms used herein have the same meaning as commonly understood by one of ordinary skill in the art to which this invention belongs.

The term “target gene,” as used herein, refers to a gene encoding a protein suitable to target in cancer therapies, i.e. a gene for which downregulation or upregulation of expression, or inhibition or potentiation of the activity of the encoded protein, has been implicated in cancer. In the context of the present invention, a target gene is a “direct target” if the antisense oligonucleotides of the invention are designed to be complementary to a region of the gene or mRNA corresponding to the gene, and an “indirect target” if the antisense oligonucleotide of the invention has a downstream or non-antisense effect on the expression of the gene. In accordance with the present invention, the direct target gene is the gene encoding thymidylate synthase.

The term “antisense oligonucleotide,” as used herein, refers to a nucleotide sequence that is complementary to a portion of a TS gene or mRNA. In the context of the present invention, the term “oligonucleotide” refers to an oligomer or polymer of ribonucleic acid (RNA), deoxyribonucleic acid (DNA), or modified versions or analogues thereof, or RNA or DNA mimetics.

The term “selectively hybridize,” as used herein, refers to the ability of a nucleic acid to bind detectably and specifically to a second nucleic acid. Polynucleotides, oligonucleotides and fragments thereof selectively hybridize to direct target nucleic acid strands under hybridization and wash conditions that minimize appreciable amounts of detectable binding to non-specific nucleic acids. High stringency conditions can be used to achieve selective hybridization conditions as known in the art and discussed herein. Typically, hybridization and washing conditions are performed at high stringency according to conventional hybridization procedures. Washing conditions are typically 1-3×SSC, 0.1-1% SDS, 50-70° C. with a change of wash solution after about 5-30 minutes.

The term “corresponds to,” as used herein with reference to nucleic acid sequences, refers to a polynucleotide sequence that is identical to all or a portion of a reference polynucleotide sequence. In contradistinction, the term “complementary to” is used herein to mean that the polynucleotide sequence is identical to all or a portion of the complement of a reference polynucleotide sequence. For illustration, the nucleotide sequence “TATAC” corresponds to a reference sequence “TATAC” and is complementary to a reference sequence “GTATA.”

The following terms are used herein to describe the sequence relationships between two or more polynucleotides: “reference sequence,” “comparison window,” “sequence identity,” “percentage of sequence identity,” and “substantial identity.” A “reference sequence” is a defined sequence used as a basis for a sequence comparison; a reference sequence may be a subset of a larger sequence, for example, as a segment of a full-length mRNA or mRNA sequence, or may comprise a complete mRNA or mRNA sequence. Generally, a reference sequence is at least 20 nucleotides in length, but may be at least 25 nucleotides in length, or at least 50 nucleotides in length. Since two polynucleotides may each (1) comprise a sequence (i.e. a portion of the complete polynucleotide sequence) that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity.

A “comparison window,” as used herein, refers to a conceptual segment of at least 20 contiguous nucleotide positions wherein a polynucleotide sequence may be compared to a reference sequence of at least 20 contiguous nucleotides and wherein the portion of the polynucleotide sequence in the comparison window may comprise additions or deletions (i.e. gaps) of 20 percent or less as compared to the reference sequence (which does not comprise additions or deletions) for optimal alignment of the two sequences. Optimal alignment of sequences for aligning a comparison window may be conducted by the local homology algorithm of Smith and Waterman (1981) Adv. Appl. Math. 2:482, by the homology alignment algorithm of Needleman and Wunsch (1970) J. Mol. Biol. 48:443, by the search for similarity method of Pearson and Lipman (1988) Proc. Natl. Acad. Sci. (U.S.A.) 85:2444, by computerized implementations of these algorithms (GAP, BESTFIT, FASTA, and TFASTA in the Wisconsin Genetics Software Package Release 7.0, Genetics Computer Group, 573 Science Dr., Madison, Wis.), or by inspection. The best alignment (i.e. resulting in the highest percentage of identity over the comparison window) generated by the various methods is then selected.

The term “sequence identity” means that two polynucleotide sequences are identical (i.e. on a nucleotide-by-nucleotide basis) over the window of comparison. The term “percentage of sequence identity” is calculated by comparing two optimally aligned sequences over the window of comparison, determining the number of positions at which the identical nucleic acid base (e.g. A, T, C, G, U, or I) occurs in both sequences to yield the number of matched positions, dividing the number of matched positions by the total number of positions in the window of comparison (i.e. the window size), and multiplying the result by 100 to yield the percentage of sequence identity.

The term “substantial identity,” as used herein, denotes a characteristic of a polynucleotide sequence, wherein the polynucleotide comprises a sequence that has at least 50 percent sequence identity, and more usually at least 60 percent sequence identity as compared to a reference sequence over a comparison window of at least 20 nucleotide positions, and frequently over a window of at least 25-50 nucleotides, wherein the percentage of sequence identity is calculated by comparing the reference sequence to the polynucleotide sequence which may include deletions or additions which total 20 percent or less of the reference sequence over the window of comparison.

Antisense Oligonucleotides of the Present Invention

Selection and Characteristics

“Targeting” an antisense ODN to a thymidylate synthase mRNA, in the context of the present invention, is a multistep process. The process usually begins with the identification of a direct target nucleic acid sequence whose function is to be modulated. This may be, for example, a cellular gene or mRNA transcribed from the gene. The targeting process also includes determination of a site or sites within this gene for the antisense interaction to occur such that the desired effect, i.e. modulation of expression of the protein encoded by the gene, will result. Once the direct target site or sites have been identified, oligonucleotides are chosen that are sufficiently complementary (i.e. hybridize with sufficient strength and specificity) to the direct target mRNA to give the desired result.

The sequences of various TS genes and mRNAs are known in the art and can be readily obtained from Genbank (maintained by the National Center for Biotechnology Information). For example, the sequence for human TS mRNA can be accessed under Accession No. X02308 [SEQ ID NO:1].

Generally, there are five regions of a gene or mRNA that may be targeted for antisense modulation: the 5′-untranslated region (5′-UTR), the translation initiation or start codon region, the open reading frame (ORF), the translation termination or stop codon region and the 3′-untranslated region (3′-UTR). Within these regions, certain sequences may be directly targeted, for example, known regulatory sequence elements (such as those for post-transcriptional control and mRNA stability) or regions that are unique to a group of mRNAs encoding similar proteins.

The terms “translation initiation codon” and “start codon” can encompass many codon sequences, even though the initiator amino acid in each instance is typically methionine in eukaryotes. It is also known in the art that eukaryotic genes may have two or more alternative start codons, any one of which may be preferentially utilized for translation initiation in a particular cell type or tissue, or under a particular set of conditions. In the context of the invention, “start codon” and “translation initiation codon” refer to the codon or codons that are used in vivo to initiate translation of an mRNA molecule transcribed from a gene encoding TS regardless of the sequence(s) of such codons.

As is known in the art, some eukaryotic transcripts are directly translated, however, most mammalian ORFs contain one or more sequences, known as “introns,” which are excised from a transcript before it is translated; the expressed (unexcised) portions of the ORF are referred to as “exons” (Alberts et al., (1983) Molecular Biology of the Cell, Garland Publishing Inc., New York, pp. 411-415). In the context of the present invention, both introns and exons may serve as targets for antisense modulation.

Thus, the antisense ODNs according to the present invention can be complementary to regions of the unprocessed mRNA of the direct target gene including the introns, or the antisense ODNs can be complementary to part of the processed mRNA of the direct target gene.

In some instances, an ORF may also contain one or more sites that may be directly targeted for antisense modulation due to some functional significance in vivo. Examples of the latter types of sites include intragenic stem-loop structures (see, for example, U.S. Pat. No. 5,512,438) and, in unprocessed mRNA molecules, intron/exon splice sites. In addition, mRNA molecules possess a 5′-cap region that may also serve as a direct target for antisense. The 5′-cap of a mRNA comprises an N⁷-methylated guanosine residue joined to the 5′-most residue of the mRNA via a 5′-5′ triphosphate linkage. The 5′-cap region of a mRNA is considered to include the 5′-cap structure itself as well as the first 50 nucleotides adjacent to the cap.

There are also elements in the 3′-UTR region which can impact upon message stability, including examples of unique cis-elements that interact with trans-acting proteins to control mRNA turnover rates (Hake and Richer, (1997) Biochim. Biophys. Acta 1332:M31-M38). In addition, the polyadenylated tail can serve several functions impacting upon translation efficiency and message turnover, for example, by protecting the message from degradation, depending on the length of the poly ‘A’ tail (Ford et al, (1997) Mol. Cell. Biol. 17:398-406).

In one embodiment of the invention, the antisense ODNs directly target the 3′-UTR region of a TS mRNA. In another embodiment of the invention, the antisense ODNs directly target the 5′-UTR region of a TS mRNA. In another embodiment, the antisense ODNs target the coding region of a TS mRNA. In still other embodiments, the antisense ODNs span the stop codon or start codon of a TS mRNA.

The antisense ODNs in accordance with the present invention are selected from a sequence complementary to the direct target gene such that the sequence exhibits the least likelihood of forming duplexes, hair-pins, or of containing homooligomer/sequence repeats. The ODN may further contain a GC clamp. One skilled in the art will appreciate that these properties can be determined qualitatively using various computer modelling programs, for example, the program OLIGO® Primer Analysis Software, Version 5.0 (distributed by National Biosciences, Inc., Plymouth, Minn.).

Alternatively, the antisense ODNs can be selected on the basis that the sequence is highly conserved for the direct target gene between two or more species. These properties can be determined using the BLASTN program (Altschul, et al., (1990) J. Mol. Biol., 215:403-10) of the University of Wisconsin Computer group (GCG) software (Devereux. et al., (1984) Nucleic Acids Res., 12:387-395) with the National Center for Biotechnology Information (NCBI) databases.

In order to be effective, antisense ODNs are typically between 7 and 100 nucleotides in length. In one embodiment of the present invention the antisense ODNs comprise from at least about 7 to about 50 nucleotides, or nucleotide analogues. In another embodiment, the antisense ODNs comprise from about 12 to about 35 nucleotides, or nucleotide analogues. In another embodiment, the antisense ODNs comprise from about 15 to about 25 nucleotides, or nucleotide analogues.

It is understood in the art that an antisense ODN need not have 100% identity with the complement of its direct target sequence. The antisense ODNs in accordance with the present invention have a sequence that is at least about 75% identical to the complement of direct target sequence. In one embodiment of the present invention, the antisense oligonucleotides have a sequence that is at least about 90% identical to the complement of the direct target sequence. In another embodiment, they have a sequence that is at least about 95% identical to the complement of direct target sequence, allowing for gaps or mismatches of several bases. Identity can be determined, for example, by using the BLASTN program of the University of Wisconsin Computer Group (GCG) software.

In accordance with the present invention, the antisense ODNs comprise at least 7 consecutive nucleotides that are complementary to the sequence of a TS mRNA. In one embodiment, the antisense ODNs comprise at least 7 consecutive nucleotides that are complementary to a human TS mRNA. In another embodiment, the antisense ODNs comprise at least 7 consecutive nucleotides that are complementary to the human TS mRNA set forth in SEQ ID NO: 1.

In order for the antisense ODNs of the present invention to function as modulators of the expression of TS mRNA, it is necessary that they demonstrate adequate specificity for the direct target sequence and do not bind to other sequences in the cell. Therefore, in addition to possessing an appropriate level of sequence identity to the complement of a TS mRNA, the antisense ODNs of the present invention should not closely resemble other known sequences. The antisense ODNs of the present invention, therefore, preferably have less than 15 nucleotides identical to any other sequence, more preferably less than 12 nucleotides identical and most preferably less than 7 nucleotides identical to any other sequence.

It will, however, be appreciated by one skilled in the art that the degree of acceptable identity between sequences may vary, for example, according to the length of the antisense ODNs and the relative position of the identical nucleotides in the sequences within a comparison window, such that greater than a 15 nucleotide identity may exist, and the antisense ODN still demonstrates adequate specificity for a direct target sequence. The identity of the antisense ODNs of the present invention to other sequences can be determined, for example, through the use of the BLASTN program and the NCBI databases as indicated above.

In one embodiment of the present invention, the antisense ODNs against TS comprise at least 5 consecutive nucleotides from one of the sequences provided in Table 1. In another embodiment, the antisense ODNs comprise at least 7 consecutive nucleotides from one of the sequences provided in Table 1. In another embodiment, the antisense ODNs comprise at least 10 consecutive nucleotides from one of the sequences provided in Table 1.

TABLE 1

Exemplary Antisense ODNs against Human TS

| | Complementary | |
|---|---|---|
| Sequence (5′ → 3′) | Region in TS mRNA | SEQ ID NO |
| GCCAGTGGCAACATCCTTAA | 1184-1203 | 2 |
| TTGGATGCGGATTGTACCCT | 1002-1021 | 3 |
| ACTCAGCTCCCTCAGATTTG | 1436-1455 | 4 |
| CCAGCCCAACCCCTAAAGAC | 1081-1100 | 5 |
| GGCATCCCAGATTTTCACTC | 419-438 | 6 |
| AGCATTTGTGGATCCCTTGA | 380-399 | 7 |

Modifications to Antisense Oligonucleotides

In the context of this invention, the term “oligonucleotide” includes oligonucleotides composed of naturally-occurring nucleobases, sugars and covalent internucleoside (backbone) linkages as well as oligonucleotides having non-naturally-occurring portions, which function similarly. Such modified or substituted oligonucleotides are often preferred over native forms because of desirable properties such as, for example, enhanced cellular uptake, enhanced affinity for the nucleic acid target and increased stability in the presence of nucleases. The term also includes chimeric oligonucleotides. Chimeric oligonucleotides are oligonucleotides that contain two or more chemically distinct regions, each region comprising at least one monomer unit. The oligonucleotides according to the present invention can be single-stranded or they can be double-stranded.

As is known in the art, a nucleoside is a base-sugar combination and a nucleotide is a nucleoside that further includes a phosphate group covalently linked to the sugar portion of the nucleoside. In forming oligonucleotides, the phosphate groups covalently link adjacent nucleosides to one another to form a linear polymeric compound, with the normal linkage or backbone of RNA and DNA being a 3′ to 5′ phosphodiester linkage. Specific examples of oligonucleotides useful in this invention include those containing modified backbones or non-natural internucleoside linkages. As defined in this specification, oligonucleotides having modified backbones include both those that retain a phosphorus atom in the backbone and those that lack a phosphorus atom in the backbone. For the purposes of the present invention, and as sometimes referenced in the art, modified oligonucleotides that do not have a phosphorus atom in their internucleoside backbone can also be considered to be oligonucleotides.

Exemplary modified oligonucleotide backbones that can be incorporated into the oligonucleotides according to the present invention include, for example, phosphorothioates, chiral phosphorothioates, bridged phosphorothioates, phosphorodithioates, phosphotriesters, aminoalkylphosphotriesters, methyl and other alkyl phosphonates including 3′-alkylene phosphonates, bridged methylene phosphonates and chiral phosphonates, phosphinates, phosphoramidates including 3′-amino phosphoramidate, bridged phosphoramidates and aminoalkylphosphoramidates, thionophosphoramidates, thionoalkylphosphonates, thionoalkylphosphotriesters, and boranophosphates having normal 3′-5′ linkages, 2′-5′ linked analogues of these, and analogues having inverted polarity wherein the adjacent pairs of nucleoside units are linked 3′-5′ to 5′-3′ or 2′-5′ to 5′-2′. Various salts, mixed salts and free acid forms are also included.

Exemplary modified oligonucleotide backbones that do not include a phosphorus atom are formed by short chain alkyl or cycloalkyl internucleoside linkages, mixed heteroatom and alkyl or cycloalkyl internucleoside linkages, or one or more short chain heteroatomic or heterocyclic internucleoside linkages. Such backbones include morpholino linkages (formed in part from the sugar portion of a nucleoside); siloxane backbones; sulphide, sulphoxide and sulphone backbones; formacetyl and thioformacetyl backbones; methylene formacetyl and thioformacetyl backbones; carbonate backbones; carboxymethylester backbones; acetamidate backbones; carbamate backbones; thioether backbones; alkene containing backbones; sulphamate backbones; methyleneimino and methylenehydrazino backbones; sulphonate and sulphonamide backbones; amide backbones; and others having mixed N, O, S and CH₂ component parts.

The term “alkyl” as used herein refers to monovalent alkyl groups having from 1 to 20 carbon atoms. In one embodiment of the present invention the alkyl group has between 1 and 6 carbon atoms. Examples of suitable alkyl groups include, but are not limited to, methyl, ethyl, n-propyl, iso-propyl, n-butyl, iso-butyl, n-hexyl, and the like.

The term “cycloalkyl” refers to cyclic alkyl groups of from 3 to 20 carbon atoms having a single cyclic ring or multiple condensed rings. Examples of suitable cycloalkyl groups include, but are not limited to, single ring structures such as cyclopropyl, cyclobutyl, cyclopentyl, cyclooctyl, and the like, or multiple ring structures such as adamantanyl, and the like.

The present invention also contemplates oligonucleotide mimetics in which both the sugar and the internucleoside linkage of the nucleotide units are replaced with novel groups. The base units are maintained for hybridization with an appropriate nucleic acid target. An example of such an oligonucleotide mimetic, which has been shown to have excellent hybridization properties, is a peptide nucleic acid (PNA) [Nielsen et al., Science, 254:1497-1500 (1991)]. In PNA compounds, the sugar-backbone of an oligonucleotide is replaced with an amide-containing backbone, in particular an aminoethylglycine backbone. The nucleobases are retained and are bound directly or indirectly to aza-nitrogen atoms of the amide portion of the backbone.

The present invention also contemplates oligonucleotides comprising “locked nucleic acids” (LNAs), which are novel conformationally restricted oligonucleotide analogues containing a methylene bridge that connects the 2′-O of ribose with the 4′-C (see, Singh et a., Chem. Commun., 1998, 4:455-456). LNA and LNA analogues display very high duplex thermal stabilities with complementary DNA and RNA, stability towards 3′-exonuclease degradation, and good solubility properties. Synthesis of the LNA analogues of adenine, cytosine, guanine, 5-methylcytosine, thymine and uracil, their oligomerization, and nucleic acid recognition properties have been described (see Koshkin et al., Tetrahedron, 1998, 54:3607-3630). Studies of mis-matched sequences show that LNA obey the Watson-Crick base pairing rules with generally improved selectivity compared to the corresponding unmodified reference strands. Antisense oligonucleotides containing LNAs have been described (Wahlestedt et al., Proc. Natl. Acad. Sci. U.S.A., 2000, 97:5633-5638), which were efficacious and non-toxic. In addition, the LNA/DNA copolymers were not degraded readily in blood serum and cell extracts.

LNAs form duplexes with complementary DNA or RNA or with complementary LNA, with high thermal affinities. The universality of LNA-mediated hybridization has been emphasized by the formation of exceedingly stable LNA:LNA duplexes (Koshkin et al., J. Am. Chem. Soc., 1998, 120:13252-13253). LNA:LNA hybridization was shown to be the most thermally stable nucleic acid type duplex system, and the RNA-mimicking character of LNA was established at the duplex level. Introduction of three LNA monomers (T or A) resulted in significantly increased melting points toward DNA complements.

Synthesis of 2′-amino-LNA (Singh et al., J. Org. Chem., 1998, 63, 10035-10039) and 2′-methylamino-LNA has been described and thermal stability of their duplexes with complementary RNA and DNA strands reported. Preparation of phosphorothioate-LNA and 2′-thio-LNA have also been described (Kumar et al., Bioorg. Med. Chem. Lett., 1998, 8:2219-2222).

Modified oligonucleotides according to the present invention may also contain one or more substituted sugar moieties. For example, oligonucleotides may comprise sugars with one of the following substituents at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Examples of such groups are: O[(CH₂)ₙ O]ₘ CH₃, O(CH₂)ₙ OCH₃, O(CH₂)ₙ NH₂, O(CH₂)ₙ CH₃, O(CH₂)ₙ ONH₂, and O (CH₂)ₙ ON[(CH₂)ₙ CH₃)]₂, where n and m are from 1 to about 10. Alternatively, the oligonucleotides may comprise one of the following substituents at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂ CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Specific examples include 2′-methoxyethoxy (2′-O—CH₂ CH₂ OCH₃, also known as 2′-O-(2-methoxyethyl) or 2′-MOE) [Martin et al., Helv. Chim. Acta, 78:486-504(1995)], 2′-dimethylaminooxyethoxy (O(CH₂)₂ ON(CH₃)₂ group, also known as 2′-DMAOE), 2′-methoxy (2′-O—CH₃), 2′-aminopropoxy (2′-OCH₂ CH₂ CH₂ NH₂) and 2′-fluoro (2′-F).

Similar modifications may also be made at other positions on the oligonucleotide, particularly the 3′ position of the sugar on the 3′ terminal nucleotide or in 2′-5′ linked oligonucleotides and the 5′ position of the 5′ terminal nucleotide. Oligonucleotides may also comprise sugar mimetics such as cyclobutyl moieties in place of the pentofuranosyl sugar.

Oligonucleotides according to the present invention may also include modifications or substitutions to the nucleobase. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C); inosine; 5-hydroxymethyl cytosine; xanthine; hypoxanthine; 2-aminoadenine; 6-methyl and other alkyl derivatives of adenine and guanine; 2-propyl and other alkyl derivatives of adenine and guanine; 2-thiouracil, 2-thiothymine and 2-thiocytosine; 5-halouracil and cytosine; 5-propynyl uracil and cytosine; 6-azo uracil, cytosine and thymine; 5-uracil (pseudouracil); 4-thiouracil; 8-halo, 8-amino, 8-thiol, 8-thioalkyl, 8-hydroxyl and other 8-substituted adenines and guanines; 5-halo particularly 5-bromo, 5-trifluoromethyl and other 5-substituted uracils and cytosines; 7-methylguanine and 7-methyladenine; 8-azaguanine and 8-azaadenine; 7-deazaguanine and 7-deazaadenine; 3-deazaguanine and 3-deazaadenine. Further nucleobases include those disclosed in U.S. Pat. No. 3,687,808; The Concise Encyclopaedia Of Polymer Science And Engineering, (1990) pp 858-859, Kroschwitz, J. I., ed. John Wiley & Sons; Englisch et al., Angewandte Chemie, Int. Ed., 30:613 (1991); and Sanghvi, Y. S., (1993) Antisense Research and Applications, pp 289-302, Crooke, S. T. and Lebleu, B., ed., CRC Press. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. [Sanghvi, Y. S., (1993) Antisense Research and Applications, pp 276-278, Crooke, S. T. and Lebleu, B., ed., CRC Press, Boca Raton].

Another oligonucleotide modification included in the present invention is the chemical linkage to the oligonucleotide of one or more moieties or conjugates which enhance the activity, cellular distribution or cellular uptake of the oligonucleotide. Such moieties include, but are not limited to, lipophilic or lipid moieties such as a cholesterol moiety [Letsinger et al., Proc. Natl. Acad. Sci. USA, 86:6553-6556 (1989)], cholic acid [Manoharan et al., Bioorg. Med. Chem. Let., 4:1053-1060 (1994)], a thioether, e.g. hexyl-S-tritylthiol [Manoharan et al., Ann. N.Y. Acad. Sci., 660:306-309 (1992); Manoharan et al., Bioorg. Med. Chem. Lett., 3:2765-2770 (1993)], a thiocholesterol [Oberhauser et al., Nucl. Acids Res., 20:533-538 (1992)], an aliphatic chain, e.g. dodecandiol or undecyl residues [Saison-Behmoaras et al., EMBO J., 10:1111-1118 (1991); Kabanov et al., FEBS Lett., 259:327-330 (1990); Svinarchuk et al., Biochimie, 75:49-54 (1993)], a phospholipid, e.g. di-hexadecyl-rac-glycerol or triethylammonium 1,2-di-O-hexadecyl-rac-glycero-3-H-phosphonate [Manoharan et al., Tetrahedron Lett., 36:3651-3654 (1995); Shea et al., Nucl. Acids Res., 18:3777-3783 (1990)], a polyamine or a polyethylene glycol chain [Manoharan et al., Nucleosides & Nucleotides, 14:969-973 (1995)], or adamantane acetic acid [Manoharan et al., Tetrahedron Lett., 36:3651-3654 (1995)], a palmityl moiety [Mishra et al., Biochim. Biophys. Acta, 1264:229-237 (1995)], or an octadecylamine or hexylamino-carbonyl-oxycholesterol moiety [Crooke et al., J. Pharmacol. Exp. Ther., 277:923-937 (1996)].

One skilled in the art will recognise that it is not necessary for all positions in a given oligonucleotide to be uniformly modified. The present invention, therefore, contemplates the incorporation of more than one of the aforementioned modifications into a single oligonucleotide or even at a single nucleoside within the oligonucleotide.

As indicated above, oligonucleotides that are chimeric compounds are included within the scope of the present invention. For purposes of the invention, a “chimeric oligonucleotide” refers to an oligonucleotide having more than one type of internucleoside linkage or comprising more than one type of modified nucleotide. Non-limiting examples include oligonucleotides having an alkylphosphonate-linked region and an alkylphosphonothioate region (as described, for example, in U.S. Pat. Nos. 5,635,377 and 5,366,878), oligonucleotides containing at least one, or more typically, at least three or four consecutive phosphodiester or phosphorothioate internucleoside linkages. Other examples of chimeric oligonucleotides include those comprising a ribonucleotide or 2′-O-substituted ribonucleotide region (for example, comprising from about 2 to about 12 2′-O-substituted nucleotides), and a deoxyribonucleotide region. Such chimeric oligonucleotides have been previously described (see, for example, U.S. Pat. Nos. 5,652,355 and 5,652,356). Inverted chimeric oligonucleotides are also contemplated, as described in U.S. Pat. Nos. 5,652,356; 5,973,136, and 5,773,601.

Particularly useful chimeric oligonucleotides are mixed backbone oligonucleotides (MBOs) which contain centrally-modified or end-modified nucleosides with appropriately placed segments of modified internucleotide linkages, such as phosphorothioates, methylphosphonates, phosphodiesters, and segments of modified oligodeoxynucleotides or oligoribonucleotides (Agrawal (1997) Proc. Natl. Acad. Sci., USA, 94:2620-2625; Agrawal (1999) Biochem. Biophys. Acta 1489:53-67).

In the context of the present invention, an oligonucleotide is “nuclease resistant” when it has either been modified such that it is not susceptible to degradation by DNA and RNA nucleases or, alternatively, has been placed in a delivery vehicle which itself protects the oligonucleotide from DNA or RNA nucleases. Nuclease resistant oligonucleotides include, for example, methyl phosphonates, phosphorothioates, phosphorodithioates, phosphotriesters, and morpholino oligomers. Suitable delivery vehicles for conferring nuclease resistance include, for example, liposomes.

The present invention further contemplates oligonucleotides that contain groups for improving the pharmacokinetic properties of the oligonucleotide, or groups for improving the pharmacodynamic properties of the oligonucleotide.

In one embodiment of the present invention, the antisense ODNs comprise at least one phosphorothioate linkage. In another embodiment, the antisense ODNs comprise at least one 2′-methoxy-ethoxy substituted nucleotide. In another embodiment, the antisense ODNs comprise a plurality of 2′-methoxy-ethoxy substituted nucleotides at both the 3′- and 5′-end of the ODN. In still another embodiment, the antisense ODNs comprise both at least one phosphorothioate linkage and a plurality of 2′-methoxy-ethoxy substituted nucleotides at both the 3′- and 5′-ends of the ODN.

Preparation of the Antisense Oligonucleotides

The antisense oligonucleotides of the present invention can be prepared by conventional techniques well-known to those skilled in the art (see, for example, U.S. Pat. No. 6,087,489). For example, the oligonucleotides can be prepared using solid-phase synthesis using commercially available equipment, such as the equipment available from Applied Biosystems Canada Inc., Mississauga, Canada. As is well-known in the art, modified oligonucleotides, such as phosphorothioates and alkylated derivatives, can also be readily prepared by similar methods.

The isolation and purification of antisense oligonucleotides can be accomplished using, for example, filtration, extraction, crystallization, different forms of chromatography, including column, thin layer, preparative low or high pressure liquid chromatography, or a combination of these procedures, in addition to other equivalent separation or isolation procedures.

Alternatively, the antisense oligonucleotides of the present invention can be prepared by enzymatic digestion of the naturally occurring direct target gene by methods known in the art.

Antisense oligonucleotides can also be prepared through the use of recombinant methods. The present invention, therefore encompasses expression vectors comprising nucleic acid sequences that encode the antisense oligonucleotides and expression of the encoded antisense oligonucleotides in a suitable host cell. Such expression vectors can be readily constructed using procedures known in the art. Examples of suitable vectors include, but are not limited to, plasmids, phagemids, cosmids, bacteriophages, baculoviruses and retroviruses, and DNA viruses. One skilled in the art will understand that selection of the appropriate host cell for expression of the antisense oligonucleotide will be dependent upon the vector chosen. Examples of host cells include, but are not limited to, bacterial, yeast, insect, plant and mammalian cells.

One skilled in the art will also understand that the expression vector may further include regulatory elements, such as transcriptional elements, required for efficient transcription of the antisense oligonucleotide sequences. Examples of regulatory elements that can be incorporated into the vector include, but are not limited to, promoters, enhancers, terminators, and polyadenylation signals. The present invention, therefore, provides vectors comprising a regulatory element operatively linked to a nucleic acid sequence encoding an antisense oligonucleotide. One skilled in the art will appreciate that selection of suitable regulatory elements is dependent on the host cell chosen for expression of the antisense oligonucleotide and that such regulatory elements may be derived from a variety of sources, including bacterial, fungal, viral, mammalian or insect genes.

In the context of the present invention, the expression vector may additionally contain a reporter gene. Suitable reporter genes include, but are not limited to, β-galactosidase, green fluorescent protein, red fluorescent protein, luciferase, and β-glucuronidase. Incorporation of a reporter gene into the expression vector allows transcription of the antisense oligonucleotide to be monitored by detection of a signal generated by expression of the reporter gene.

In accordance with the present invention, the expression vectors can be introduced into a suitable host cell or tissue by one of a variety of methods known in the art. Such methods can be found generally described in Sambrook et al., Molecular Cloning: A Laboratory Manual, Cold Spring Harbor Laboratory, New York (1992); Ausubel et al., Current Protocols in Molecular Biology, John Wiley and Sons, Baltimore Md. (1989); Chang et al., Somatic Gene Therapy, CRC Press, Ann Arbor Mich. (1995); Vega et al., Gene Targeting, CRC Press, Ann Arbor, Mich. (1995); and Vectors: A Survey of Molecular Cloning Vectors and Their Uses, Butterworth's, Boston Mass. (1988) and include, for example, stable or transient transfection, lipofection, electroporation, and infection with recombinant viral vectors.

Testing of the Antisense Oligonucleotides

In accordance with the present invention, the antisense ODNs are selected for their ability to modulate TS expression and for their ability to modulate the expression of at least one other indirect target gene.

In Vitro Testing

The effectiveness of the antisense ODNs of the present invention in inhibiting TS expression can be demonstrated initially in vitro using, for example, the method previously described by Choy et al., 1998, Cancer Res. 48: 6949-6952.

For example, the antisense ODN can be introduced into a cell line that normally expresses TS or which over-expresses TS (for example, a tumour cell line) and the amount of mRNA transcribed from the TS gene can be measured by standard techniques such as Northern blot analysis. Alternatively, the amount of TS protein produced by the cell can be measured by standard techniques such as Western blot analysis. The amount of mRNA or protein produced in a cell treated with the antisense ODN can then be compared with the amount produced in control cells and will provide an indication of how successfully the antisense ODN has inhibited TS gene expression. Suitable control cells include untreated cells and cells treated with a control, scrambled ODN.

Antisense ODNs in accordance with the invention are those ODNs which decrease the levels of TS mRNA and/or TS protein expression when compared to untreated cells, or cells treated with a control oligonucleotide.

The antisense ODNs of the present invention can be screened for their ability to modulate the expression of genes other than the TS gene using standard methods for screening expression of multiple genes (“expression profiling”). Such methods are well known in the art and include, for example, the microarray assay described in the Examples, or high density microarray assays containing 10-fold more (for example, 19,000) human genes to identify suitable functional clusters of genes whose expression is affected by the antisense ODNs.

Typically, expression profiling makes use of pre-fabricated microarrays of short DNA sequences or oligonucleotides. Microarrays comprise an ordered arrangement of thousands of oligonucleotides, each capable of specifically hybridising to a certain gene, immobilised onto a suitable solid support. Typically microarrays useful for this purpose represent between 1,000 and 40,000 genes. Methods of constructing microarrays are well known in the art [see, for example, Ausubel, et al., Current Protocols in Molecular Biology, John Wiley & Sons, Inc, NY. (1989 and updates)]. In addition, custom-made microarrays are available from many companies. Pre-made microarrays are also commercially available for many organisms including, for example, GeneChip® (Affimetrix, Santa Clara, Calif.), Atlas™ (BD Biosciences-CLONTECH, Palo Alto, Calif.), GEM Microarrays, GeneJet™ array and LifeSeq® (Incyte Genomics, Palo Alto, Calif.), MICROMAX™ Human cDNA Microarray Systems (PerkinElmer Life Sciences, Boston, Mass.) and ResGen™ GeneFilters® (Invitrogen, Huntsville, Ala.).

For expression analysis, RNA is isolated from cells treated with the antisense ODN and from control cells. If necessary, the RNA can be amplified by conventional techniques to ensure a sufficient quantity for analysis. The RNA is then hybridised to the microarray under suitable conditions and a routine analysis of the microarray by commercially available scanners and software is conducted to identify genes whose expression is altered in the treated cells relative to the control cells. Suitable hybridization conditions can readily be determined by one skilled in the art using standard techniques.

Following the identification of such other genes, mRNA quantitation and respective protein levels can also be evaluated to determine the extent of the effect of the antisense ODN on the indirect target gene and thereby select appropriate antisense ODNs.

The ability of the antisense ODNs to inhibit cancer cell growth can also be tested in vitro. For example, the colony forming ability or proliferation of neoplastic cells treated with the antisense ODNs can be tested by growing neoplastic cells to an appropriate density (e.g. approximately 1×10⁴) and then adding an appropriate concentration of one or more antisense ODN in the presence of cationic lipid (e.g. lipofectin to a final concentration of 5 μg/mL). Excess antisense ODN is washed away after a suitable incubation period and the cells are subsequently cultured using standard techniques. Percent inhibition of colony forming ability or proliferation is calculated by comparison of the number of colonies in the treated culture with the number of colonies in control cultures, for example, cultures not pre-treated with antisense ODNs, those pre-treated with a control, scrambled ODN or those treated with a standard chemotherapeutic (as visualized, for example, by methylene blue staining). Similar methods can be employed to determine the effect of the antisense ODNs in combination with one or more chemotherapeutic.

In Vivo and Ex Vivo Testing

The efficacy of the antisense ODNs of the present invention can be tested in vivo in an appropriate animal model. In general, current animal models for screening anti-tumour compounds are xenograft models, in which a human tumour has been implanted into an animal. Examples of xenograft models of human cancer include, but are not limited to, human solid tumour xenografts in mice, implanted by sub-cutaneous injection and used in tumour growth assays; human solid tumour isografts in mice, implanted by fat pad injection and used in tumour growth assays; experimental models of lymphoma and leukaemia in mice, used in survival assays; and experimental models of lung metastasis in mice. Examples of currently accepted xenograft models are provided in Table 2.

Antisense ODNs alone or in combination with one or more chemotherapeutic may be administered to neoplastic cells ex vivo prior to injection of the cells into the mice or they may be administered to the mice in vivo after the injection of the cells and tumour establishment in the mice.

When administered in vivo, the antisense oligonucleotides of the present invention can be administered to the animal by, for example, systemic administration (e.g. tail vein injection) or local administration, e.g. into a tumour. Alternatively, the antisense ODNs can be administered by continuous spinal delivery, for example, via an intratheal catheter attached to a mini-osmotic pump.

As an example, the antisense ODN in combination with one or more chemotherapeutic can be tested in vivo on solid tumours using mice that are subcutaneously grafted bilaterally with a pre-determined amount of a tumour fragment on day 0. The animals bearing tumours are mixed before being subjected to the various treatments and controls. In the case of treatment of advanced tumours, tumours are allowed to develop to the desired size, animals having insufficiently developed tumours being eliminated. The selected animals are distributed at random into groups that will undergo the treatments or act as controls. Suitable groupings would be, for example, those receiving the antisense ODN and the one or more chemotherapeutic, those receiving the antisense ODN alone, those receiving the chemotherapeutic agent(s) alone and those receiving no treatment or treatment with a control, scrambled ODN. Animals not bearing tumours may also be subjected to the same treatments as the tumour-bearing animals in order to be able to dissociate the toxic effect from the specific effect on the tumour. Chemotherapy generally begins from 3 to 22 days after grafting, depending on the type of tumour, and the animals are observed every day. The antisense ODNs of the present invention can be administered to the animals, for example, by bolus infusion. The different animal groups are weighed about 3 or 4 times a week until the maximum weight loss is attained, after which the groups are weighed at least once a week until the end of the trial.

The tumours are measured about 2 or 3 times a week until the tumour reaches a pre-determined size and/or weight, or until the animal dies if this occurs before the tumour reaches the pre-determined size/weight. The animals are then sacrificed and the tissue histology, size and/or proliferation of the tumour assessed.

For the study of the effect of the antisense ODN with one or more chemotherapeutic on leukaemias, the animals are grafted with a particular number of cells, and the anti-tumour activity is determined by the increase in the survival time of the treated mice relative to the controls.

To study the effect of the antisense ODN with one or more chemotherapeutic on tumour metastasis, tumour cells are typically treated ex vivo and then injected into a suitable test animal. The spread of the tumour cells from the site of injection is then monitored over a suitable period of time by standard techniques.

In vivo toxic effects of the oligonucleotides can be evaluated by measuring their effect on animal body weight during treatment and by performing haematological profiles and liver enzyme analysis after the animal has been sacrificed.

TABLE 2
Examples of Xenograft Models of Human Cancer

| Cancer Model | Cell Type |
|---|---|
| Tumour Growth Assay | Prostate (PC-3, DU145) |
| Human solid tumour xenografts | Breast (MDA-MB-231, MVB-9) |
| in mice (sub-cutaneous | Colon (HT-29) |
| injection) | Lung (NCI-H460, NCI-H209) |
| | Pancreatic (ASPC-1, SU86.86) |
| | Pancreatic: drug resistant (BxPC-3) |
| | Skin (A2058, C8161) |
| | Cervical (SIHA, HeLa-S3) |
| | Cervical: drug resistant (HeLa S3-HU- |
| | resistance) |
| | Liver (HepG2) |
| | Brain (U87-MG) |
| | Renal (Caki-1, A498) |
| | Ovary (SK-OV-3) |
| Tumour Growth Assay | Breast: drug resistant (MDA-CDDP-S4, |
| Human solid tumour isografts in | MDA-MB435-To.1) |
| mice (fat pad injection) | |
| Survival Assay | Human: Burkitts lymphoma (Non- |
| Experimental model of | Hodgkin's) (raji) |
| lymphoma and leukaemia | Murine: erythroleukemia (CB7 Friend |
| in mice | retrovirus-induced) |
| Experimental model of lung | Human: melanoma (C8161) |
| metastasis in mice | Murine: fibrosarcoma (R3) |

Studies to assess the toxicology, pharmacokinetics and bioavailability of chemotherapeutics when administered in combination with the antisense ODNs of the present invention can also be conducted using techniques known on the art (see, for example, Maines et al., (eds.) Current Protocols in Toxicology, J. Wiley & Sons, New York, N.Y.; “Pharmacokinetics Processes and Mathematics”, Welling, P. E., ACS Monograph 185; 1986).
Applications for the Antisense Oligonucleotides
Cancer Therapeutics

In accordance with the present invention, the antisense ODNs to TS provide for improved cancer therapies.

The antisense ODNs of the present invention can be used to sensitise tumour cells to the action of one or more chemotherapeutic agents (i.e. enhance the effects of standard dosages of the chemotherapeutics). In particular, the antisense ODNs can be used to sensitise tumour cells to the action of one or more chemotherapeutics that target either, or both, of TS and the indirect target. Alternatively, the antisense ODNs can be used to sensitise tumour cells to the action of one or more chemotherapeutics that are normally broken down or catabolised in vivo by the protein encoded by indirect target gene. Such antisense ODNs can also be used to increase the bioavailability of the one or more chemotherapeutic. In one embodiment of the present invention, the antisense ODNs sensitise tumour cells to the effects of fluoropyrimidine-based chemotherapeutics.

The antisense ODNs of the invention may also be used to decrease the effective dosage of a particular chemotherapeutic agent that needs to be administered to a patient during a chemotherapy regimen. Alternatively, or in addition, the antisense ODNs may be used to decrease the side effects of a chemotherapeutic agent. In one embodiment of the invention, the antisense ODNs are used with less than standard dosages of the chemotherapeutic in the treatment of various cancers.

It is contemplated that antisense ODNs directed against TS mRNA also have application against advanced neoplastic disease (i.e. overt disease in a patient that is not amenable to cure by local modalities of treatment, such as surgery or radiotherapy). The use of the antisense ODNs in combination with standard chemotherapeutics at less than standard dosage can allow for more prolonged treatments as may be necessary to treat advanced disease. It is also contemplated that the antisense ODNs have application as part of adjuvant therapies where the intention is to cure the cancer in a patient.

The antisense ODNs can also be used in combination with one or more chemotherapeutic agents to improve cancer therapies in drug resistant cancers which can arise, for example, from heterogeneity of tumour cell populations, alterations in response to chemotherapy and increased malignant potential. Such changes are often more pronounced at advanced stages of disease and have, in part, as an underlying cause, changes in genome/message stability.

The antisense ODNs of the present invention are useful as drugs for the treatment of cancer or proliferative disorders irrespective of their origin. Cancers which may be treated using the methods of the invention, include, but are not limited to carcinomas, leukemias (e.g. of the central-nervous system and blood), lymphomas (Hodgkins and non-Hodgkins), sarcomas, melanomas, adenomas, neuroblastomas, nephroblastomas (e.g. Wilm's tumour) and retinoblastomas.

Examples of carcinomas (i.e. cancers originating in epithelial tissues such as the skin and inner membrane surfaces of the body), include, but are not limited to cancers such as breast cancer, colon cancer, rectal cancer, esophageal cancer, prostate cancer, lung cancer, stomach cancer, bladder cancer, skin cancer, kidney cancer, pancreatic cancer, ovarian cancer, uterine cancer, cervical cancer, cancer of the vulva, liver cancer, thyroid cancer, aveolar cell carcinoma, basal cell carcinoma, bronchogenic carcinoma, chorionic carcinoma, embyronal carcinoma, giant cell carcinoma, glandular carcinoma, medullary carcinoma, melatonic carcinoma, mucinous carcinoma, oat cell carcinoma, scirrhous carcinoma and squamous cell carcinoma.

Examples of sarcomas (i.e. cancers originating in soft tissues of mesenchymal origin such as the connective and supportive tissues of muscle, bone, cartilage and fat), include, but are not limited to cancers such as Kaposi's sarcoma, alveolar soft part sarcoma, bone cancer, botryoid sarcoma, endometrial sarcoma, giant cell sarcoma, osteogenic sarcoma, reticulum cell sarcoma and spindle cell sarcoma, rhabdomyosarcoma and lymphosarcoma.

In the treatment of cancer, the antisense ODNs are used in conjunction with one or more chemotherapeutic agent, particularly for the treatment of cancers suspected of being drug resistant. Chemotherapeutic agents (e.g. synthetic chemical medications) have toxic effects that selectively or non-selectively destroy cancerous tissue. A variety of chemotherapeutics are suitable for use in conjunction with the antisense ODNs of the invention. One important group of chemotherapeutic agents contemplated by the present invention is the group of chemotherapeutics targeting TS. As is known in the art, TS is frequently overexpressed in a wide variety of neoplastic cells, therefore, chemotherapeutics against TS mRNA can be used in the treatment of a range of cancers. Examples of other chemotherapeutics that can be used in conjunction with the antisense ODNs of the invention include, but are not limited to, hydroxyurea, busulphan, cisplatin, carboplatin, chlorambucil, melphalan, cyclophosphamide, Ifosphamide, danorubicin, doxorubicin, epirubicin, mitoxantrone, vincristine, vinblastine, Navelbine® (vinorelbine), etoposide, teniposide, paclitaxel, docetaxel, gemcitabine, cytosine, arabinoside and the like. The antisense ODNs are also suitable for use with standard combination therapies employing two or more chemotherapeutic agents.

Standard dosage and administration regimens for chemotherapeutic agents are well known in the art and can be found, for example, in the product monographs published in the Compendium of Pharmaceuticals and Specialties, 31^{st} Edition, 1996 (CPS), or the latest edition thereof.

For example, Navelbine® indicated for the treatment of cancer or tumours, such as breast cancer and non-small cell lung cancer, and the dosage and administration protocol suggested in the product monograph in the CPS is 30 mg/m², administered weekly via intravenous injection over 6 to 10 minutes. No dose adjustment is required for patients with renal insufficiency. Adjustment to dosage is suggested in accordance with hematologic toxicity or hepatic insufficiency.

In one embodiment of the present invention, the antisense ODNs are used in conjunction with one or more chemotherapeutic agent that targets TS. TS inhibiting drugs are typically fluoropyrimidine-based drugs. Examples of TS inhibiting chemotherapeutics include, but are not limited to, the fluoropyrimidine drugs 5-FU, 5-FUdR, capecitabine (an oral form of a pro-drug of 5-FU) and a topical 5-FU cream (Effudex®), as well as the non-fluoropyrimidine drugs raltitrexed, methotrexate and Alimta® (pemetrexed)

5-FU has been used as chemotherapeutic for many years alone and in conjunction with other chemotherapeutics. The following exemplary therapeutic regimens are provided with the understanding that one skilled in the art would appreciate that they may be applied to the situations where 5-FU is used alone or conjunction with another chemotherapeutic. A first exemplary regimen is the Mayo regimen, wherein 1 cycle consists of 5-FU administered at 425 mg/m² by intravenous bolus injection daily together with 20 mg/m² leucovorin for 5 days, followed by 3 weeks off. A second therapeutic regimen may consist of administering 200 to 220 mg/m² 5-FU by continuous infusion over 24 hours once a week. A third therapeutic regimen consists of shorter, intermittent infusions of 5-FU from between 24 to 120 hours, every week, two weeks, three weeks or four weeks at dosages of 600 mg/m² to 2500 mg/m² per 24 hours. One skilled in the art will also appreciate that 5-FU and its variants can be used in combination therapies with a variety of other traditional chemotherapeutic drugs.

An exemplary therapeutic regimen for raltitrexed (Tomudex®) is administration at 3 mg/m² once every 3 weeks by bolus injection.

As indicated above, chemotherapeutics are generally administered using a particular therapeutic regimen over a period of weeks or months, but can have deleterious effects on healthy tissues, for example, by suppressing the bone marrow to some degree or lowering white blood cell counts, resulting in increased risk of infection for patients due to immunosuppression. In addition, many chemotherapeutics can result in a variety of side-effects over the course of the regimen. For example, continuous 5-FU infusion can lead to hand-foot syndrome, mucositis, diarrhoea, nausea and vomiting (Leonard, 2001, British J. Cancer, 84:1437-1442).

Continuous infusion therapy can be labour intensive, inconvenient for the patient and is associated with complications and additional costs related to the need for central venous access. Improved versions of current chemotherapeutics that are suitable for oral administration are therefore continually being sought. One example of a chemotherapeutic developed for oral administration is capecitabine, which is a prodrug form of 5-FU. An example of a therapeutic regimen for oral administration of capecitabine (Xeloda®) consists of two divided doses for a total of 2000-2500 mg/m² daily. Oral administration of capecitabine, however, is typically associated with undesirable and dose limiting side-effects, in particular hand-foot syndrome.

While a few compounds exhibiting lessened toxic effects with equal or greater chemotherapeutic activity have been achieved, for example, 3′,4′-anhydrovinblastine as compared to other vinca alkaloids, the process of synthesizing such chemical medicines and screening them for activity can be labour and time intensive. Thus a need remains for relatively non-toxic drugs that can be produced efficiently and provide improved anti-tumour efficacy for the treatment of cancer, or otherwise potentiate the effect of, or decrease the side-effects of, known chemotherapeutics. As indicated above, the present invention provides for a method of reducing some of the deleterious effects of chemotherapeutic agents using the antisense ODNs of the invention by virtue of their ability to modulate expression of the indirect target gene(s). Antisense ODNs are generally less toxic than standard chemotherapeutics, for example, the TS antisense ODN shown in SEQ ID NO: 2 is known not to exhibit overt toxicity in animals (Berg et al., 2001, J. Pharmacol. Exp. Ther. 298:477-484).

In addition to toxic effects, certain chemotherapeutics are subject to breakdown or catabolism in the body, which leads to greatly reduced bioavailability. For example, the pharmacokinetics of 5-FU have been studied in some detail and indicate that, while the conversion of the drug to nucleotide derivatives is responsible for most of its antineoplastic activity, breakdown of the drug via the uracil degradation pathway accounts for more than 80% of the administered dose of the drug (Diasio and Harris, 1989, Clin. Pharmacokinet., 16:215-237).

Dehydropyrimidine dehydrogenase (DPD) is the primary rate-limiting enzyme in the catabolism of fluorouracil. When the level of DPD activity is low (normalized for normal tissue values) in a tumour, there is a higher likelihood of tumour response and, therefore, lowering the activity further would enhance the therapeutic activity of the 5-FU. In addition, the variability of fluorouracil pharmacokinetics has been attributed to the activity of DPD, in that inhibition or inactivation of DPD can eliminate this variability and render the effects of fluorouracil treatments more predictable. The catabolites produced by the breakdown of fluorouracil are associated with a number of toxicities that can be dose-limiting. Inhibition of DPD, therefore, can lead to a decrease in those toxicities associated with the use of 5-FU, in particular, hand-foot syndrome, cardiotoxicity and certain neurotoxicities (Diasio, 1998, Oncology, 12:23-27), which may in turn lead to the requirement for decreased dosages of 5-FU.

DPD is particularly active in the intestine and, as a result, fluoropyrimidine-based therapeutics are typically administered by continuous infusion. Inhibitors of DPD have been developed, for example, eniluracil (Glaxo Wellcome), uracil and 5-chloro-2,4-dihydropyrimidine (CDHP), which can be used in combination with 5-FU or its variants to permit oral administration of the drug. Examples of such combinations include, UFT (Orzel, Bristol-Myers Squibb), which contains tegafur (ftoraphor) plus uracil; S-1 (Taiho, Bristol-Myers Squibb), which contains CDHP and a prodrug of fluorouracil, and BOF-A2 (Otsuka). Capecitabine, being a prodrug form of 5-FU that is resistant to DPD activity, circumvents DPD breakdown in the intestine and is thus suitable for oral administration.

In one embodiment of the present invention, the indirect target of the antisense ODN is a DPD gene. In another embodiment, the antisense ODN acts on a indirect target DPD gene to inhibit expression of DPD. Antisense ODNs of the present invention, for which an indirect target is DPD, can be used in conjunction with fluoropyrimidine-based chemotherapeutics to potentiate the activity of these compounds in the treatment of cancer and/or to minimise one or more dose-limiting toxicities of the chemotherapeutic. Alternatively, the antisense ODN can be used in conjunction with a fluoropyrimidine-based chemotherapeutic and a DPD inhibitor to potentiate the action of the inhibitor.

As indicated above, combinations of fluoropyrimidine-based chemotherapeutics and DPD inhibitors are particularly useful as they can be administered orally. In one embodiment of the present invention, therefore, the one or more fluoropyrimidine-based chemotherapeutic with or without a DPD inhibitor is administered orally and the antisense ODN is administered parenterally. In another embodiment, the one or more fluoropyrimidine-based chemotherapeutic with or without a DPD inhibitor and the antisense ODN are formulated for oral administration.

Methods of Identifying Potential Targets for Cancer Therapies

The antisense ODNs of the present invention can be used to identify potential targets for cancer therapies. Treatment of cells with the antisense ODNs and subsequent expression analysis using techniques such as those described above, can provide information about indirect target genes whose expression is affected by the antisense ODNs. Such indirect target genes are potential targets in the development of new chemotherapeutics or cancer treatment strategies.

Pharmaceutical Preparations Antisense Oligonucleotides

When employed as pharmaceuticals, the antisense oligonucleotides are usually administered in the form of pharmaceutical compositions or formulations. Such compositions are prepared in a manner well known in the pharmaceutical art and comprise at least one active compound. In a related embodiment, the pharmaceutical composition or formulation comprises a vector encoding the antisense oligonucleotide of the present invention.

In accordance with the present invention, the antisense oligonucleotides may be incorporated into pharmaceutical compositions in the form of pharmaceutically acceptable salts. The term “pharmaceutically acceptable salt” as used herein refers to salts which retain the biological effectiveness and properties of the antisense oligonucleotides of the present invention, and which are not biologically or otherwise undesirable. In many cases, the antisense oligonucleotides of the present invention are capable of forming acid and/or base addition salts by virtue of the presence of amino and/or carboxyl groups or groups similar thereto.

Pharmaceutically acceptable base addition salts can be prepared from inorganic and organic bases. Salts derived from inorganic bases include, but are not limited to, sodium, potassium, lithium, ammonium, calcium and magnesium salts. Salts derived from organic bases include, but are not limited to, salts of primary, secondary and tertiary amines, such as alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, cycloalkyl amines, di(cycloalkyl) amines, tri(cycloalkyl) amines, substituted cycloalkyl amines, disubstituted cycloalkyl amine, trisubstituted cycloalkyl amines, cycloalkenyl amines, di(cycloalkenyl) amines, tri(cycloalkenyl) amines, substituted cycloalkenyl amines, disubstituted cycloalkenyl amine, trisubstituted cycloalkenyl amines, aryl amines, diaryl amines, triaryl amines, heteroaryl amines, diheteroaryl amines, triheteroaryl amines, heterocyclic amines, diheterocyclic amines, triheterocyclic amines, mixed di- and tri-amines where at least two of the substituents on the amine are different and are selected from the group consisting of alkyl, substituted alkyl, alkenyl, substituted alkenyl, cycloalkyl, substituted cycloalkyl, cycloalkenyl, substituted cycloalkenyl, aryl, heteroaryl, heterocyclic, and the like. Amines in which two or three substituents, together with the amino nitrogen, form a heterocyclic or heteroaryl group are also suitable.

Examples of suitable amines include, but are not limited to, isopropylamine, trimethyl amine, diethyl amine, tri(iso-propyl) amine, tri(n-propyl) amine, ethanolamine, 2-dimethylaminoethanol, tromethamine, lysine, arginine, histidine, caffeine, procaine, hydrabamine, choline, betaine, ethylenediamine, glucosamine, N-alkylglucamines, theobromine, purines, piperazine, piperidine, morpholine, N-ethylpiperidine, and the like. It should also be understood that carboxylic acid derivatives would be useful in the practice of this invention, for example, carboxylic acid amides, including carboxamides, lower alkyl carboxamides, dialkyl carboxamides, and the like.

Pharmaceutically acceptable acid addition salts can be prepared from inorganic and organic acids. Salts derived from inorganic acids include hydrochloric acid, hydrobromic acid, sulphuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulphonic acid, ethanesulphonic acid, p-toluene-sulphonic acid, salicylic acid, and the like.

Administration of the Antisense Oligonucleotides

The antisense ODNs of the present invention and pharmaceutical compositions comprising the antisense ODNs may be administered in a number of ways depending upon whether local or systemic treatment of the organism is desired. Administration may be pulmonary, e.g. by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g. intrathecal or intraventricular, administration. The antisense oligonucleotides of the present invention and pharmaceutical compositions comprising same may be administered topically in a lotion or cream, for application to the skin in order to treat for example a melanoma.

The antisense oligonucleotides of the present invention may be delivered with a pharmaceutically acceptable vehicle. Ideally, such a vehicle would enhance the stability and/or delivery properties. The present invention also provides for administration of the antisense oligonucleotides or pharmaceutical compositions comprising the antisense oligonucleotides using a suitable vehicle, such as a liposome, microparticle or microcapsule. In various embodiments of the invention, the use of such vehicles may be beneficial in achieving sustained release of the active component, or otherwise protecting the antisense ODNs from nuclease degradation.

For administration to an individual for the treatment cancer, the present invention also contemplates the formulation of the antisense oligonucleotides or pharmaceutical compositions comprising the antisense oligonucleotides into oral dosage forms such as tablets, capsules and the like. For this purpose, the antisense oligonucleotides or pharmaceutical compositions comprising the antisense oligonucleotides can be combined with conventional carriers, such as magnesium carbonate, magnesium stearate, talc, sugar, lactose, pectin, dextrin, starch, gelatin, tragacanth, methylcellulose, sodium carboxymethyl-cellulose, low melting wax, cocoa butter and the like. Diluents, flavoring agents, solubilizers, lubricants, suspending agents, binders, tablet-disintegrating agents and the like can also be employed, if required. The antisense oligonucleotides or pharmaceutical compositions comprising the antisense oligonucleotides can be encapsulated with or without other carriers. In all cases, the proportion of active ingredients in any solid and liquid composition will be at least sufficient to impart the desired activity to the individual being treated upon oral administration. The present invention further contemplates parenteral injection of the antisense oligonucleotides or pharmaceutical compositions comprising the antisense oligonucleotides, in which case they are used in the form of a sterile solution containing other solutes, for example, enough saline or glucose to make the solution isotonic.

For administration by inhalation or insufflation, the antisense oligonucleotides or pharmaceutical compositions comprising the antisense oligonucleotides can be formulated into an aqueous or partially aqueous solution, which can then be utilized in the form of an aerosol.

The present invention also provides for administration of the antisense oligonucleotides in the form of genetic vector constructs that are designed to direct the in vivo synthesis of the antisense oligonucleotides. Within the vector construct, the nucleic acid sequence encoding the antisense oligonucleotide is under the control of a suitable promoter. The vector construct may additionally contain other regulatory control elements. Methods of constructing and administering such genetic vector constructs for in vivo synthesis of antisense oligonucleotides are well-known in the art. U.S. Pat. No. 6,265,167 teaches an efficient method for the introduction, expression and accumulation of antisense oligonucleotides in the cell nucleus. This method allows the antisense oligonucleotide to hybridize to the sense mRNA in the nucleus, and thereby prevents the antisense oligonucleotide being either processed or transported into the cytoplasm.

The dosage requirements for the antisense oligonucleotides of the present invention or pharmaceutical compositions comprising the antisense oligonucleotides vary with the particular compositions employed, the route of administration, the severity of the symptoms presented and the particular subject being treated. Dosage requirements can be determined by standard clinical techniques, known to a worker skilled in the art. Treatment will generally be initiated with small dosages less than the optimum dose of the compound. Thereafter the dosage is increased until the optimum effect under the circumstances is reached. In general, the antisense oligonucleotides or pharmaceutical compositions comprising the antisense oligonucleotides are administered at a concentration that will generally afford effective results without causing any harmful or deleterious side effects. Administration can be either as a single unit dose or, if desired, the dosage can be divided into convenient subunits that are administered at suitable times throughout the day.

In one embodiment of the present invention, the antisense ODNs are administered systemically to patients, for example, by bolus injection or continuous infusion into a patient's bloodstream. In another embodiment, the antisense ODNs are administered by continuous intravenous infusion.

When used in conjunction with one or more chemotherapeutic agent, the antisense ODN can be administered prior to, or after, administration of the one or more chemotherapeutic agents, or it can be administered concomitantly. The one or more chemotherapeutic may be administered systemically, for example, by bolus injection or continuous infusion, or it may be administered orally.

When the antisense ODN is administered prior to the one or more chemotherapeutic agents, the length of time between administration of the antisense ODN and chemotherapeutic will depend on the mode of administration and the size of the patient. In one embodiment of the present invention, the one or more chemotherapeutic agent is administered within 4 to 24 hours of treatment with the antisense ODN.

When the antisense ODN and the one or more other chemotherapeutic agents are administered concurrently, administration of the compounds may be initiated at the same time, or administration of the other chemotherapeutic(s) may be initiated at a suitable time after administration of the antisense ODN was initiated.

Therapeutic Uses and Strategies

The antisense oligonucleotides of the invention may be used as part of a neo-adjuvant therapy (to primary therapy), as part of an adjuvant therapy regimen, or also for the treatment of locally advanced and metastatic diseases.

Primary therapy is understood to encompass a first line of treatment upon the initial diagnosis of cancer in a patient. Exemplary primary therapies may involve surgery, a wide range of chemotherapies and radiotherapy.

Adjuvant therapy is understood to encompass any therapy, following a primary therapy such as surgery, that is administered to patients at risk of relapsing. Adjuvant systemic therapy is begun soon after primary therapy to delay recurrence, prolong survival or cure a patient. One kind of adjuvant systemic therapy is adjuvant chemotherapy, e.g., using 5-fluorouracil alone or in combination with methotrexate for breast and colorectal cancers, over the course of e.g., four to 24 months. It is contemplated that the antisense ODNs can be used in further combination with other chemotherapeutic agents as part of an adjuvant therapy.

In the application of cancer therapies a patient's response status is monitored. Response status refers to measuring what happens to the tumour(s) or lesion(s) under chemotherapy, namely any observed growth (progression of disease), stability, or shrinkage (complete or partial response). Arising out such monitoring may be the observation of relapse in a patient which may refer to the relapse of a patient with advanced disease. Relapse time is the time from the initial appearance of a primary cancer to the appearance of advanced disease requiring chemotherapy.

The progression of advanced disease is monitored to help evaluate when chemotherapy may be appropriate and may be marked by an increase of at least 25% in the overall sum of measurable lesions as compared to nadir (ie. best response) and/or the appearance of new lesions following primary therapy. Alternatively, lesions may be found to shrink in size.

To gain a better understanding of the invention described herein, the following examples are set forth. It should be understood that these examples are for illustrative purposes only. Therefore, they should not limit the scope of this invention in any way.

### EXAMPLES

Oligodeoxynucleotides

A fully phosphorothioated ODN with 2′-methoxy-ethoxy modification on the 6 nucleotides at both the 5′- and 3′-ends was generously provided by ISIS Pharmaceuticals (Carlsbad, Calif.): ODN SEQ ID NO: 2 (5′-GCCAGTGGCAACATCCTTAA-3′).

The scrambled control ODN SEQ ID NO: 8 (5′-ATGCGCCAACGGTTCCTAAA-3′) has the same base composition as ODN SEQ ID NO: 2, in random order, and is not complementary to any region of human TS. There are no other known human mRNAs with more than 15 bases of complementarity to any of the ODNs used. The ODNs were diluted in Milli-Q purified water, and concentrations calculated based on spectrophotometric absorbance readings.

### Example 1

### Microarray Assay

To test the effect of ODN SEQ ID NO: 2 on the expression of genes (other than the gene encoding for TS) HeLa cells were treated with 100 nM TS antisense ODN SEQ ID NO: 2 or scrambled control ODN SEQ ID NO: 8 using 1 μg/ml LipofectAmine 2000™ (InVitrogen). Cells were collected at various times (8, 16, 24 and 48 hours) and RNA isolated using TriZol™. Reverse-transcribed cDNA was labelled with Cy3- or Cy5-dCTP, using CyScribe™ (Amersham). Microarrays containing 1716 human genes were obtained from the Ontario Microarray Consortium (Toronto, Canada). Hybridization was completed at 42° C. overnight, followed by high stringency washes at 60° C. with 0.1×SSC, 0.2% SDS. Slides were scanned on a ChipReader™ scanner (Virtek Vision Inc., Waterloo, Canada) and signals were quantitated with ArrayVision™ (Imaging Research, Inc. St. Catherines, Canada). Data was analyzed using GeneSpring™ version 4.1.5 (Silicon Genetics, Redwood City, Calif., USA).

The effects of ODNs may be evaluated in a similar manner for a range of doses, for example, 25 to 100 nM, for a panel of ODNs, for ODNs in combination with one or more chemotherapeutics, or using other cell lines, such as HT-29 cells.

Results

The above experimental protocol was used to analyze the downstream gene expression changes following TS antisense ODN treatment. HeLa cells were treated in vitro with TS antisense ODN SEQ ID NO: 2 or control scrambled ODN SEQ ID NO: 8 for 24 and 48 hours and RNA purified. Microarrays containing 1716 human ESTs were hybridized with Cy3-and Cy5-labelled cDNA corresponding to the mRNA isolated from treated and control cells in reciprocal experiments (4 slides per time-point). Preliminary analysis of the results revealed that expression of glyceraldehyde 3-phosphate dehydrogenase, phosphoglycerate kinase, and subunit 2 of RNA polymerase II were essentially unchanged by ODN SEQ ID NO: 2 treatment at 24 hours (expression of 1.632, 1.198 and 0.806, respectively) and 48 hours (1.508, 0.774 and 0.927, respectively). Equivalent expression of ODN SEQ ID NO: 8-treated and ODN SEQ ID NO: 2-treated cells at both time points was observed for 216 analyzable genes. TS expression was suppressed at 24 and 48 hours (0.727 and 0.313, respectively) in HeLa cells treated with ODN SEQ ID NO: 2 versus ODN SEQ ID NO: 8. RT-PCR confirmed down-regulation of TS mRNA relative to GAPDH following treatment with TS antisense ODN SEQ ID NO: 2 but not the controlled scrambled ODN SEQ ID NO: 8. Among the genes that were repressed by ODN SEQ ID NO: 2 treatment compared to ODN SEQ ID NO: 8 treatment, dihydropyrimidine dehydrogenase and protein tyrosine phosphatase-1B were down-regulated at 24 hours (0.823 and 0.112, respectively) or 48 hours (0.438 and 0.542, respectively). On the other hand, increased expression of c-myb (1.767-fold) histone H3B (2.034-fold) and cytokeratin 8 (1.589-fold) was measured at 24 hours after ODN SEQ ID NO: 2 treatment, and 1.752-, 1.547- and 1.858-fold increases were determined for these same three genes, respectively, in the 48 hour samples.

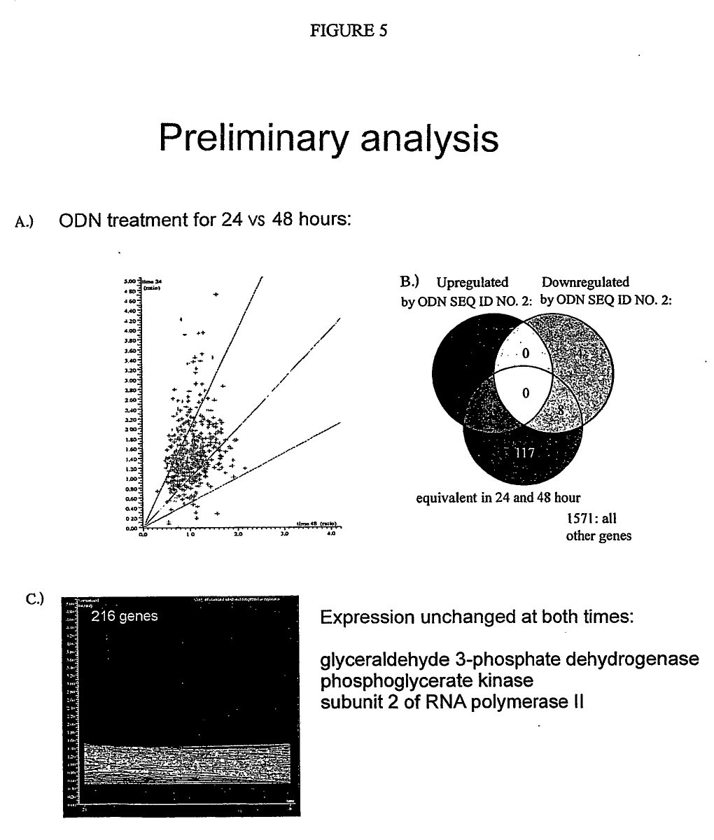

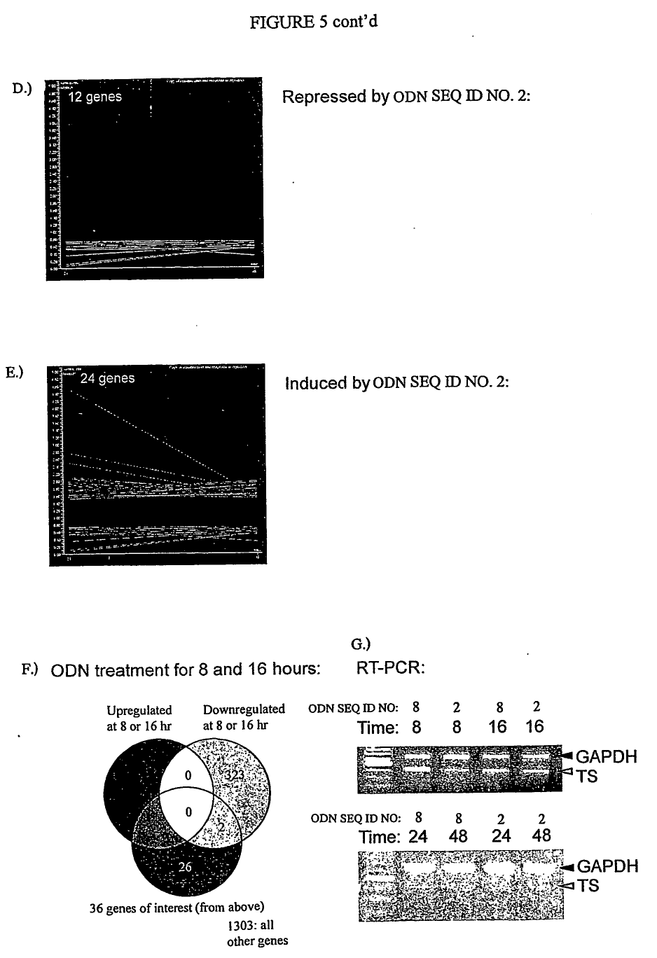

FIG. 5A shows the results on gene expression in HeLa cells exposed to TS antisense ODN treatment at 24 versus 48 hours. Equivalent gene expression was seen in 216 analyzable genes in the presence of ODN SEQ ID NO: 2 (TS-antisense) and ODN SEQ ID NO: 8 (scrambled control ODN). FIGS. 5B to E show that TS antisense ODN SEQ ID NO: 2 treatment repressed expression of at least 12 genes including TS, dihydropyrimidine dehydrogenase, adenylate cyclase type I, casein kinase I alpha, protein tryrosine phosphatase 1B and DNA polymerase alpha. ODN SEQ ID NO: 2 treatment induced the expression of at lease 24 genes including transcription factor SOX-4, cellular apoptosis susceptibility protein, MAP kinase activated protein kinase 2, c-myb proto-oncogene, cytokeratin 8 and histone H3B. At 8 and 16 hours following treatment with ODN SEQ ID NO: 2 many other genes besides the ones described above were either repressed or induced, however, the analysis of which genes these are is ongoing (FIG. 5F). A time course of the effects of ODN SEQ ID NO: 2 on HeLa cell TS mRNA levels is shown in FIG. 5G. Treatment of HeLa cells with ODN SEQ ID NO: 2 dramatically decreased levels of TS mRNA at 24 and 48 hours post treatment, however, the scrambled control ODN SEQ ID NO: 8 had similar TS mRNA levels at the 8, 16, 24 and 48 hour time points. The mRNA levels for GAPDH were unchanged in the presence of either ODN SEQ ID NO: 2 or ODN SEQ ID NO: 8.

Summary

Microarray assays to evaluate global gene expression changes occurring in HeLa cells following TS antisense ODN treatment demonstrate for the first time that ODNs complementary to TS mRNA (e.g. SEQ ID NO: 2) have downstream effects on other genes, inducing the upregulation or downregulation of a number of human genes encoding for proteins which are known targets, or potential targets, for cancer therapy.

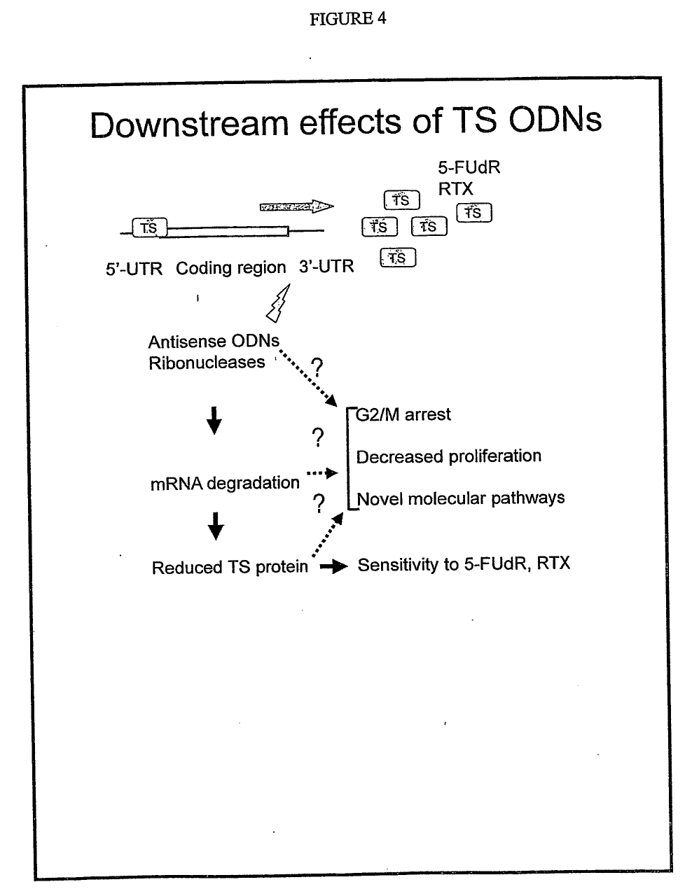

FIG. 4 shows TS down-regulation by antisense ODN treatment activates novel molecular pathways. A logical pathway can be followed from antisense ODN treatment through reductions in TS mRNA and protein to increased sensitivity to 5-Fluorouracil and raltitrexed. G₂/M arrest induced by ODN treatment and disconnections between these downstream effects upon treatment with a panel of TS antisense ODNs suggest that additional functions of TS mRNA or protein, or other non-TS mediated functions, are being affected by antisense treatment.

ODNs of the invention exhibiting such downstream or non-antisense effects can be in the design of more effective cancer therapies, for example, as part of therapeutic regimen comprising these ODNs.

The embodiments of the invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

The embodiments of the invention in which an exclusive property or privilege is claimed are defined as follows:

1. A composition comprising:
(a) an antisense oligonucleotide, or analogue thereof, comprising at least 7 consecutive nucleotides complementary to a thymidylate synthase mRNA;
(b) a fluoropyrimidine-based chemotherapeutic, and optionally
(c) a pharmaceutically acceptable carrier or diluent,
wherein said antisense oligonucleotide or analogue thereof, inhibits expression of a thymidylate synthase gene and modulates the expression of at least one other gene.

2. The composition according to claim 1, wherein said fluoropyrimidine-based chemotherapeutic is incorporated into said composition at less than standard dosage.

3. The composition according to claim 1 or 2, wherein said antisense oligonucleotide, or analogue thereof, inhibits expression of one or more of said at least one other gene.

4. The composition according to claim 3, wherein one or more of said at least one other gene encodes dihydropyrimidine dehydrogenase (DPD).

5. The composition according to claim 4, further comprising a DPD inhibitor.

6. The composition according to any one of claims 1 to 5, wherein said antisense oligonucleotide, or analogue thereof, comprises at least 7 consecutive nucleotides complementary to the sequence as set forth in SEQ ID NO:1.

7. The composition according to any one of claims 1 to 5, wherein said antisense oligonucleotide, or analogue thereof, comprises at least 7 consecutive nucleotides complementary to the sequence as set forth in SEQ ID NO:2.

8. The composition according to any one of claims 1 to 5, wherein said antisense oligonucleotide, or analogue thereof, comprises the sequence as set forth in SEQ ID NO:2.

9. Use of the composition according to claim 4 or 5, to potentiate the effect of a DPD inhibitor in a mammal in need thereof.

10. Use of an antisense oligonucleotide, or analogue thereof, comprising at least 7 consecutive nucleotides complementary to a thymidylate synthase mRNA to sensitise neoplastic cells to a chemotherapeutic agent, wherein said antisense oligonucleotide, or analogue thereof, inhibits expression of a thymidylate synthase gene and modulates the expression of at least one other gene.

11. The use according to claim 10, wherein said chemotherapeutic agent is a fluoropyrimidine-based chemotherapeutic agent.

12. The use according to claim 10 or 11, wherein said antisense oligonucleotide, or analogue thereof, inhibits expression of one or more of said at least one other gene.

13. The use according to claim 12, wherein said at least one other gene-encodes dihydropyrimidine dehydrogenase.

14. The use according to any one of claims 10 to 13, wherein said antisense oligonucleotide, or analogue thereof, comprises at least 7 consecutive nucleotides complementary to the sequence as set forth in SEQ ID NO:1.

15. The use according to any one of claims 10 to 13, wherein said antisense oligonucleotide, or analogue thereof, comprises at least 7 consecutive nucleotides complementary to the sequence as set forth in SEQ ID NO:2.

16. Use of an antisense oligonucleotide, or analogue thereof, comprising at least 7 consecutive nucleotides complementary to a thymidylate synthase mRNA in combination with one or more chemotherapeutic agent in the treatment of cancer in a mammal in need thereof, wherein said antisense oligonucleotide, or analogue thereof, inhibits expression of a thymidylate synthase gene and modulates the expression of at least one other gene, and wherein said one or more chemotherapeutic agent is used at less than standard dosage.

17. The use according to claim 16, wherein said one or more chemotherapeutic agent is a fluoropyrimidine-based chemotherapeutic agent.

18. The use according to claim 16 or 17, wherein said antisense oligonucleotide, or analogue thereof, inhibits expression of one or more of said at least one other gene.

19. The use according to claim 18, wherein said at least one other gene encodes dihydropyrimidine dehydrogenase.

20. The use according to any one of claims 16 to 19, wherein said antisense oligonucleotide, or analogue thereof, comprises at least 7 consecutive nucleotides complementary to the sequence as set forth in SEQ ID NO:1.

21. The use according to any one of claims 16 to 19, wherein said antisense oligonucleotide, or analogue thereof, comprises at least 7 consecutive nucleotides complementary to the sequence as set forth in SEQ ID NO:2.

22. Use of an antisense oligonucleotide, or analogue thereof, comprising at least 7 consecutive nucleotides complementary to a thymidylate synthase mRNA to increase the bioavailability of a fluoropyrimidine-based chemotherapeutic in a mammal in need thereof, wherein said antisense oligonucleotide, or analogue thereof, inhibits expression of a thymidylate synthase gene and modulates the expression of at least one other gene.

23. The use according to claim 22, wherein said antisense oligonucleotide, or analogue thereof, inhibits expression of one or more of said at least one other gene.

24. The use according to claim 23, wherein said at least one other gene encodes dihydropyrimidine dehydrogenase.

25. The use according to any one of claims 22 to 24, wherein said antisense oligonucleotide, or analogue thereof, comprises at least 7 consecutive nucleotides complementary to the sequence as set forth in SEQ ID NO:1.

26. The use according to any one of claims 22 to 24, wherein said antisense oligonucleotide, or analogue thereof, comprises at least 7 consecutive nucleotides complementary to the sequence as set forth in SEQ ID NO:2.

27. Use of an antisense oligonucleotide, or analogue thereof, comprising at least 7 consecutive nucleotides complementary to a thymidylate synthase mRNA to decrease one or more dose-limiting toxicities of a fluoropyrimidine-based chemotherapeutic in a mammal in need thereof, wherein said antisense oligonucleotide, or analogue thereof, inhibits expression of a thymidylate synthase gene and modulates the expression of at least one other gene.

28. The use according to claim 27, wherein said antisense oligonucleotide, or analogue thereof, inhibits expression of one or more of said at least one other gene.

29. The use according to claim 28, wherein said at least one other gene encodes dihydropyrimidine dehydrogenase.

30. The use according to claim 29, wherein said one or more dose limiting toxicities is hand-foot syndrome, a cardiotoxicity or a neurotoxicity.

31. The use according to claim 29, wherein said one or more dose limiting toxicities is hand-foot syndrome.

32. The use according to any one of claims 27 to 31, wherein said antisense oligonucleotide, or analogue thereof, comprises at least 7 consecutive nucleotides complementary to the sequence as set forth in SEQ ID NO:1.

33. The use according to any one of claims 27 to 31, wherein said antisense oligonucleotide, or analogue thereof, comprises at least 7 consecutive nucleotides complementary to the sequence as set forth in SEQ ID NO:2.

34. Use of an antisense oligonucleotide, or analogue thereof, comprising at least 7 consecutive nucleotides complementary to a thymidylate synthase mRNA to potentiate the effect of a dihydropyrimidine dehydrogenase inhibitor in a mammal in need thereof, wherein said antisense oligonucleotide, or analogue thereof, inhibits expression of a thymidylate synthase gene and the expression of a dihydropyrimidine dehydrogenase gene.

35. The use according to claim 34, wherein said antisense oligonucleotide, or analogue thereof, comprises at least 7 consecutive nucleotides complementary to the sequence as set forth in SEQ ID NO:1.

36. The use according to claim 34, wherein said antisense oligonucleotide, or analogue thereof, comprises at least 7 consecutive nucleotides complementary to the sequence as set forth in SEQ ID NO:2.

37. Use of an antisense oligonucleotide, or analogue thereof, comprising at least 7 consecutive nucleotides complementary to SEQ ID NO: 2 to inhibit the expression of a dihydropyrimidine dehydrogenase gene in a mammal in need thereof.

38. A method of screening for potential drug targets for cancer therapy comprising the steps of:
(a) contacting a first population of cancer cells with an antisense oligonucleotide, or analogue thereof, comprising at least 7 consecutive nucleotides complementary to a thymidylate synthase mRNA;
(b) isolating mRNA from said cancer cells to provide a treated mRNA sample;
(c) isolating mRNA from a second control population of cells to provide a control mRNA sample;
(d) conducting gene expression assays using said treated mRNA sample and said control mRNA sample to determine genes whose expression is modulated in the first population of cancer cells, thereby identifying potential drug targets for cancer therapy.

39. The method according to claim 38, wherein said antisense oligonucleotide, or analogue thereof, comprises at least 7 consecutive nucleotides complementary to the sequence as set forth in SEQ ID NO:1.

40. The method according to claim 38, wherein said antisense oligonucleotide, or analogue thereof, comprises at least 7 consecutive nucleotides complementary to the sequence as set forth in SEQ ID NO:2.


SEQUENCE LISTING (8 sequences)

SEQ ID NO: 1 - DNA, 1536 bp, Homo sapiens
ggggggggggggaccacttggcctgcctccgtcccgccgcgccacttggcctgcctccgtcccgccgcgccacttcgcctgcctccgtcccccgcccgccgcgccatgcctgtggccggctcggagctgccgcgccggcccttgccccccgccgcacaggagcgggacgccgagccgcgtccgccgcacggggagctgcagtacctggggcagatccaacacatcctccgctgcggcgtcaggaaggacgaccgcacgggcaccggcaccctgtcggtattcggcatgcaggcgcgctacagcctgagagatgaattccctctgctgacaaccaaacgtgtgttctggaagggtgttttggaggagttgctgtggtttatcaagggatccacaaatgctaaagagctgtcttccaagggagtgaaaatctgggatgccaatggatcccgagactttttggacagcctgggattctccaccagagaagaaggggacttgggcccagtttatggcttccagtggaggcattttggggcagaatacagagatatggaatcagattattcaggacagggagttgaccaactgcaaagagtgattgacaccatcaaaaccaaccctgacgacagaagaatcatcatgtgcgcttggaatccaagagatcttcctctgatggcgctgcctccatgccatgccctctgccagttctatgtggtgaacagtgagctgtcctgccagctgtaccagagatcgggagacatgggcctcggtgtgcctttcaacatcgccagctacgccctgctcacgtacatgattgcgcacatcacgggcctgaagccaggtgactttatacacactttgggagatgcacatatttacctgaatcacatcgagccactgaaaattcagcttcagcgagaacccagacctttcccaaagctcaggattcttcgaaaagttgagaaaattgatgacttcaaagctgaagactttcagattgaagggtacaatccgcatccaactattaaaatggaaatggctgtttagggtgctttcaaaggagcttgaaggatattgtcagtctttaggggttgggctggatgccgaggtaaaagttctttttgctctaaaagaaaaaggaactaggtcaaaaatctgtccgtgacctatcagttattaatttttaaggatgttgccactggcaaatgtaactgtgccagttctttccataataaaaggctttgagttaactcactgagggtatctgacaatgctgaggttatgaacaaagtgaggagaatgaaatgtatgtgctcttagcaaaaacatgtatgtgcatttcaatcccacgtacttataaagaaggttggtgaatttcacaagctatttttggaatatttttagaatattttaagaatttcacaagctattccctcaaatctgagggagctgagtaacaccatcgatcatgatgtagagtgtggttatgaactttatagttgttttatatgttgctataataaagaagtgttctgc

SEQ ID NO: 2 - DNA, 20 bp, Artificial Sequence
  antisense oligonucleotide
gccagtggcaacatccttaa

SEQ ID NO: 3 - DNA, 20 bp, Artificial Sequence
  antisense oligonucleotide
ttggatgcggattgtaccct

SEQ ID NO: 4 - DNA, 20 bp, Artificial Sequence
  antisense oligonucleotide
actcagctccctcagatttg

SEQ ID NO: 5 - DNA, 20 bp, Artificial Sequence
  antisense oligonucleotide
ccagcccaacccctaaagac

SEQ ID NO: 6 - DNA, 20 bp, Artificial Sequence
  antisense oligonucleotide
ggcatcccagattttcactc

SEQ ID NO: 7 - DNA, 20 bp, Artificial Sequence
  antisense oligonucleotide
agcatttgtggatcccttga

SEQ ID NO: 8 - DNA, 20 bp, Artificial Sequence
  antisense oligonucleotide
atgcgccaacggttcctaaa